(19) United States
(12) Patent Application Publication
(10) Pub. No.: US 2011/0040241 A1
(43) Pub. Date: Feb. 17, 2011

(54) BLOOD ACCESS ASSEMBLY FOR ARTIFICIAL LUNG AND RIGHT VENTRICULAR ASSIST DEVICE

(76) Inventors: Dongfang Wang, Lexington, KY (US); Joseph B. Zwischenberger, Lexington, KY (US)

Correspondence Address: KING & SCHICKLI, PLLC, 247 NORTH BROADWAY, LEXINGTON, KY, 40507, US

(21) Appl. No.: 12/539,110

(22) Filed: Aug. 11, 2009

(51) Int. Cl. A61M 5/00; A61M 1/00

(52) U.S. Cl. 604/28; 604/30; 604/9; 604/43

(57) ABSTRACT

A system for providing access to at least one heart chamber for delivering and/or removing a fluid includes a selectively occludable conduit defining a lumen for providing access to an interior of at least one heart chamber from an exterior of a chest wall of the patient, and a dual lumen cannula adapted for passing through an interior lumen of the conduit. The selectively occludable conduit includes at least one balloon closure for substantially sealing an end of the conduit in fluid communication with the chamber of the heart. The dual lumen cannula includes a first, drainage lumen having at least one aperture for removing a fluid from the interior of the heart chamber, and a second, delivery lumen for delivering a fluid into the interior of the heart chamber. The delivery lumen may include an extendable infusion sleeve for delivering a fluid into an interior of the heart.

### TECHNICAL FIELD

The present invention relates generally to the field of medical devices. In particular, the invention relates to cardiac medical devices and systems for use in artificial lung (AL) and right ventricular assist device (RVAD) applications.

### BACKGROUND OF THE INVENTION

It is known to provide assemblies for removing blood from a patient for oxygenation and removal of carbon dioxide via an artificial lung, and/or passing blood through an artificial heart-like device such as a right ventricular assist device to support the pumping function of a failing heart. Such assemblies utilize various cannula designs to remove blood from the patient and to return blood to the patient. In particular, it is known to provide various designs of dual lumen cannulae, whereby oxygen-depleted blood is removed via the first lumen, and oxygenated blood is returned via the second lumen. Conventional cannulae for use in such blood access assemblies during extracoiporeal blood oxygenation and like procedures typically are implanted in a peripheral blood vessel such as the jugular vein.

Disadvantageously, such systems are unsuited for long term, repeated use. Rather, during use, patient mobility is restricted and at the end of a treatment session, the cannula must be removed. In the event of subsequent treatment sessions, another cannula must be implanted, requiring multiple instances of cannula implantation and removal, with associated discomfort, trauma, and potential issues of infection to the patient. Thus, there remains a need in the art for novel devices, and methods and systems incorporating them, for providing routine, repeated access to an interior of the heart for procedures such as right ventricular assist procedures, artificial lung procedures, and the like, provided over a longer time period.

Alternatively, there are situations wherein access through the heart via the chest wall is not desirable. For example, such a surgical procedure may represent an untenable risk to a particular patient. Further, there are situations wherein an extreme surgical procedure such as accessing the heart via the chest wall, while one potential option, is undesirable due to the short-term nature of the problem to be solved. As an example, in the event of left heart failure, a left heart assist device may be implanted to provide support to one or more of the left chambers of the heart. In such situations, it is common, once the problem with the left side of the heart is ameliorated, for the right side of the heart to experience failure. This tends to occur one or two days after the left heart procedure is completed. Typically, with use of an RVAD, the problem with the right side of the heart can be corrected in approximately a week. In such situations, while it is possible to open the patient's chest to install a support device such as an RVAD, the surgery is extensive and requires extended recovery time. In such situations where providing short-term assistance to the blood pumping function of the heart is needed, percutaneous access to the heart interior may be more desirable. The present disclosure provides also improved cannula designs for accomplishing this goal.

### SUMMARY OF THE INVENTION

The present invention provides a system for accessing an interior of a heart, intended to allow routine repeated treatment sessions. The system of the invention comprises a selectively occludable conduit and a dual lumen cannula. In one aspect of the invention, a conduit is passed through the chest wall and through the wall of the right atrium (or other chamber) of the heart of a patient. The conduit is anastomosed to the skin of the chest wall on a first end, and to the right atrium on a second end. A closure mechanism is provided in an interior of the conduit to prevent leakage of blood. In the one embodiment, the closure is an occlusion balloon arrangement which may be deflated when the conduit is required to be open, and which may be inflated to occlude the conduit lumen and prevent blood leakage, or partially inflated to prevent leakage around an exterior of a cannula passed through the conduit.

A cannula having an introducer held within a central lumen thereof may then be passed through the conduit (with the balloon closure at least partially deflated) to introduce the cannula into the right atrium, after which the introducer may be withdrawn. Similarly, the occlusion balloon arrangement prevents leakage of blood at the site of anastomosis of the conduit to the right atrium of the heart.

In another aspect of the invention, cannulae for use with the described conduit are provided, defining a first lumen (drainage lumen) for withdrawal of blood from the patient and delivery of that blood to an extracoiporeal device, and a second lumen (infusion lumen) for delivery of blood from the extracoiporeal device. In one embodiment, the dual lumen cannula includes a selectively flexible portion for redirecting an extendable infusion sleeve of the second lumen to deliver blood or other fluids to a blood vessel. The flexible portion may be any suitably flexible polymer, or may include an element formed of a shape memory alloy material which, when subjected to a temperature change, such as by application of an electrical stimulus, by exposure to body temperature, or by exposure to a chilled fluid such as sterile saline, contracts and bends to the desired shape. This embodiment is contemplated for use in a right ventricular assist device (RVAD).

In another embodiment, the second, infusion lumen is configured for passing through a heart septum to access the interior of the left atrium, for use with an artificial lung (AL). This embodiment may be used in patients with such compromised lung function that it is necessary to bypass pulmonary circulation entirely until a suitable donor match can be found for a lung transplant.

These and other embodiments, aspects, advantages, and features of the present invention will be set forth in the description which follows, and in part will become apparent to those of ordinary skill in the art by reference to the following description of the invention and referenced drawings or by practice of the invention. The aspects, advantages, and features of the invention are realized and attained by means of the instrumentalities, procedures, and combinations particularly pointed out in the appended claims. Various patent and non-patent citations are discussed herein. Unless otherwise indicated, any such citations are specifically incorporated by reference in their entirety into the present disclosure.

### BRIEF DESCRIPTION OF THE DRAWINGS

The accompanying drawings incorporated in and forming a part of the specification, illustrate several aspects of the present invention, and together with the description serve to explain the principles of the invention. In the drawings:

FIGS. 1a-d show a side cross-sectional view of a selectively occludable conduit according to the present invention (FIG. 1a), a side cross-sectional view of a dual lumen cannula according to the present invention (FIG. 1b), a side cross-sectional view of a dual lumen cannula with an extended infusion sleeve (FIG. 1c), and an end cross-sectional view of the dual lumen cannula shown in FIGS. 1b-c;

FIG. 2 shows the dual lumen cannula of FIG. 1 with an introducer in place;

FIG. 3 shows the selectively occludable conduit of FIG. 1a implanted in the chest wall and heart of a patient:

FIG. 4 shows the dual lumen cannula of FIG. 1 and introducer of FIG. 2 passed through the conduit implanted as in FIG. 3;

FIG. 5 shows the dual lumen cannula of FIG. 4 with the introducer removed and the flexible portion of an infusion lumen assuming a predetermined angle;

FIG. 6 shows the dual lumen cannula of FIG. 5, with the extendable infusion sleeve extended from the infusion lumen into an interior of the pulmonary artery for a right ventricular assist function; and

FIG. 7 shows an alternative embodiment of the dual lumen cannula of FIG. 1, wherein the infusion lumen is advanced through the atrial septum into the left atrium for an artificial lung function.

### DETAILED DESCRIPTION OF THE ILLUSTRATED EMBODIMENTS

In the following detailed description of the illustrated embodiments, reference is made to the accompanying drawings that form a part hereof, and in which is shown by way of illustration, specific embodiments in which the invention may be practiced. These embodiments are described in sufficient detail to enable those skilled in the art to practice the invention. Also, it is to be understood that other embodiments may be utilized and that process, reagent, software, and/or other changes may be made without departing from the scope of the present invention.

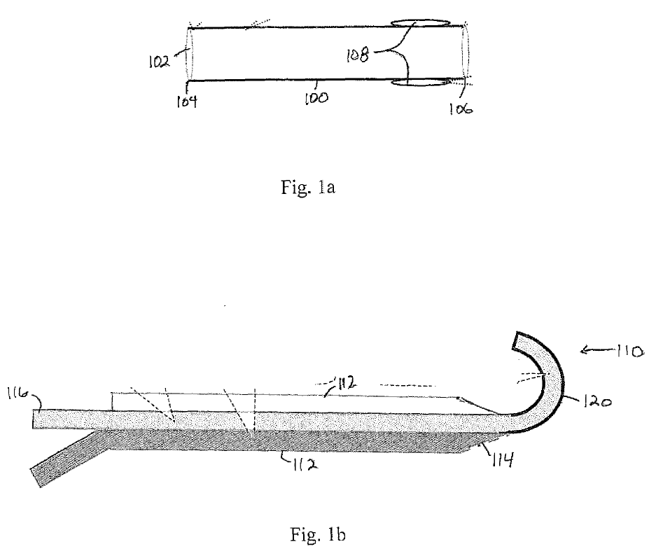

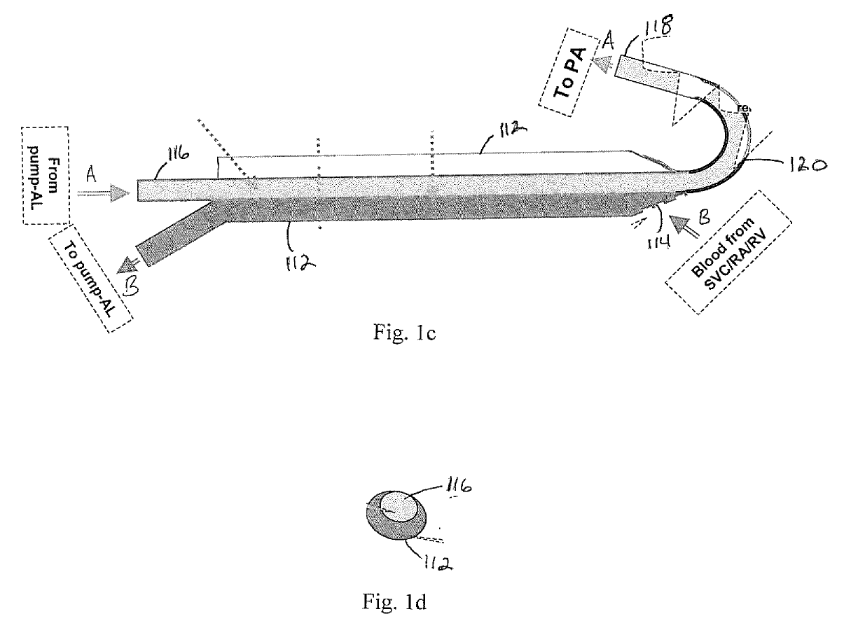

In one aspect, the present invention provides a system 10 (see FIGS. 1a-d) for providing access to a heart interior for delivering and/or removing a fluid. With reference to the drawings, FIG. 1a shows a selectively occludable conduit 100, defining an interior lumen 102 for providing access to an interior of at least one heart chamber (not shown in this figure) from an exterior of a chest wall of a patient (not shown in this Figure). The selectively occludable conduit 100 includes a first end 104 adapted to be anastomosed to the skin of a chest wall of the patient, and a second end 106 adapted to be anastomosed to the wall of a right atrium of the heart (not shown in this figure). As non-limiting examples, first end 104 and second end 106 may include one or more of suturing flanges and/or apertures (not shown for convenience) providing a plurality of attachment points for such anastomosis. The selectively occludable conduit 100 further includes an occlusion device such as an occlusion balloon 108, to substantially seal the end 106 of the conduit 100 anastomosed to die right atrium wall. It will be appreciated that this provides a mechanism for preventing bleeding and/or fluid leakage during use or between uses of the system 10.

The system 10 of the invention further includes a dual lumen cannula 110, adapted for passing through the interior lumen 102 of the conduit 100 to provide access to an interior of a heart, or for providing percutaneous access to an interior of the heart via a blood vessel, to allow delivering a fluid into and removing a fluid from the interior of the heart. Typically, the dual lumen cannula 110 defines a first, drainage lumen 112 for for establishing fluid communication with an interior of the heart for removing a fluid from the interior of the heart, and a second, delivery lumen 116 for establishing fluid communication with an interior of the heart for delivering a fluid into the interior of the heart.

The first lumen 112 is typically manufactured to provide a desired degree of stiffness to allow passing same through the conduit 100 described above, or through a lumen of a blood vessel, without sacrificing the required degree of flexibility. Any number of materials suitable for fabricating the first lumen 112 to provide the desired combination of flexibility and stiffness are known to the skilled artisan in this field. Still further, the first lumen 112 may include a surrounding reinforcing material or an integral reinforcing material (not shown for convenience) to provide the desired degree of stiffness and to prevent kinking. Suitable reinforcing materials which may be incorporated on or within the materials from which first lumen 112 is fabricated include without limitation wire, wire mesh, reinforcing polymers, and the like.

With reference to FIG. 1b, a draining end of first lumen 112 defines at least one sloped drainage opening 114, defining a transition portion between an outer surface of the first lumen 112 and an outer surface of the second lumen 116 for facilitating advancement of the cannula 110 through the above-described conduit 100 or through a lumen of a blood vessel (not shown). The skilled artisan will appreciate that the sloped drainage opening 114 may define a wide range of angles from the vertical. The drainage opening 114 may include an aperture, a perforated surface, or any structure suitable for allowing passage of a fluid to an interior of first lumen 112 for removal.

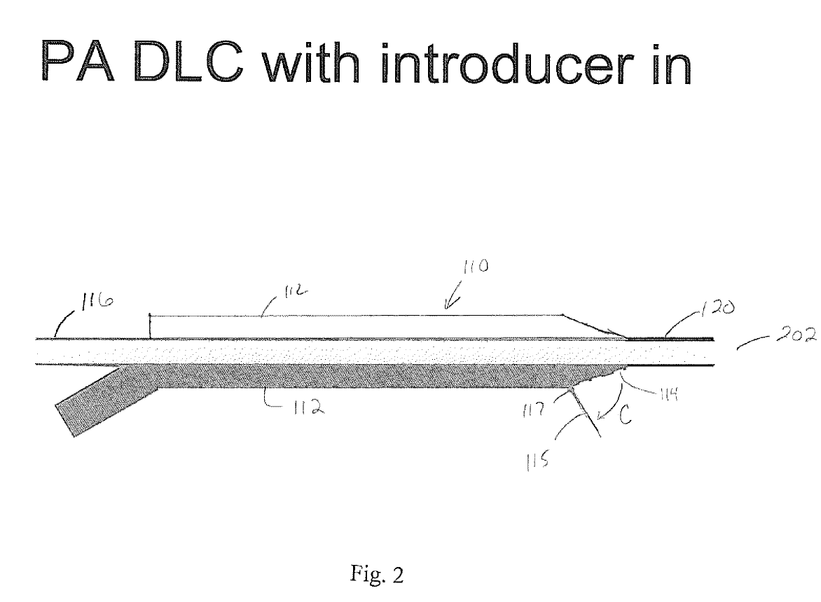

A translatable closure 115 (see FIG. 2) may be provided to occlude drainage opening 114 to substantially prevent entry of fluid into first lumen 112, such as during the process of advancing the dual lumen cannula 110 through the conduit 100 or through a blood vessel (not shown). The translatable closure 115 may be selectively translated from a closed to an open configuration (see arrow C) by any suitable means. As a non-limiting example, closure 115 may be hingedly connected to an end of drainage opening 114, whereby upon release of a catch or application of a suitable stimulus, closure 115 translates from a closed position occluding drainage opening 114 to an open position. Any number of suitable hinged connections 117 are contemplated, such as for example a flexibly polymer, spring steel, or the like. A wire or filament (not shown) may retain the closure 115 in the closed position. Once the dual lumen cannula 110 is advanced to the desired position (described in greater detail below), the wire or filament may be removed, allowing closure 115 to translate to the open position. Alternatively, the hinged connection 117 may be fabricated at least in part of a shape memory alloy, whereby application of a suitable stimulus such as an electrical current, saline held at a predetermined temperature, or the like, causes the shape memory alloy to contract, translating closure 115 to the open position.

In the depicted embodiment, the first lumen 112 and the second lumen 116 are arranged whereby at least a portion of the second lumen 116 is disposed in an interior of the first lumen 112. The second lumen 116 may be connected to the first lumen 112 along a length thereof, whereby at least a portion of the second lumen 116 is supported by the first lumen 112 (see FIG. 1d). Alternatively, the first lumen 112 and the second lumen 116 may be substantially coextensive along at least a portion of a longitudinal axis thereof, and separated by a sidewall or septum (not shown).

A distal end of the second lumen 116 terminates a distance beyond the draining end of the first lumen 112. In one embodiment of the invention, the second lumen 116 is provided with a selectively flexible portion 120 extending beyond the draining aperture 114 of the first lumen 112. This selectively flexible portion 120 may be induced to selectively assume a predetermined angle or curvature. The skilled artisan will appreciate that any number of structures or mechanisms may be employed to provide that flexible portion 120, and such structures or mechanisms are contemplated for use herein. For example, it is known to provide a spring elbow to create a predetermined angle or curvature in a lumen of a cannula, which elbow is kept in a straightened configuration by an introducer 202 (see FIG. 2) sleeved in an interior of the cannula lumen. Upon removal of the introducer 202 from an interior of the lumen, the spring elbow assumes a predetermined shape or angle as shown in FIG. 1b. Such spring elbows may be manufactured of any suitable material, such as a suitably flexible polymer, spring steel or other suitably flexible metal, and the like.

Similarly, it is known to provide a selectively flexible spring elbow (not shown in this figure) comprising a shape memory alloy such as nickel-titanium (NiTi). When kept at a first temperature, the NiTi sleeve remains in a substantially straightened position. However, upon exposure to a temperature change, such as by an electrical stimulus, body temperature, or sterile saline at an appropriate temperature, the NiTi sleeve or segment contracts to assume the predetermined shape or angle. Such devices for providing bends or predetermined angles/curvatures in cannulae are known in the art (see, for example, U.S. Pat. No. 7,090,659). The predetermined angle is intended to alter a direction of travel of the extendable infusion sleeve 118 to a sufficient degree to deliver an end of the extendable infusion sleeve 118 into an interior of a blood vessel, allowing delivery of a fluid into that blood vessel while a fluid is removed from a different portion of the interior of the heart via the first lumen 112. This will be described in detail below.

The second lumen 116 may also include at least a portion which is fabricated at least in part from a suitable membrane material, a portion of which defines an infusion sleeve 118 for delivering a fluid such as oxygenated blood into an interior of the heart. That infusion sleeve 118 is extendable at least beyond the apertures 114 disposed at the draining end of the first lumen 112 (see FIG. 1c). In one embodiment, the extendable infusion sleeve 118 is simply a length of the suitable membrane material defining a lumen, which when not in use is disposed in an interior of the second lumen 116, such as in an interior of the selectively flexible portion 120, by rolling or folding therein. Frictional contact caused by passage of a fluid through the second lumen 116 causes the extendable infusion sleeve 118 to extend out of the selectively flexible portion 120. When positioned as described above, the extendable infusion sleeve 118 is dislodged by unrolling or unfolding, and extends into an interior of the blood vessel for delivery of the fluid thereinto.

When coupled to one or more pumps and tubing as are known in this art, fluids such as oxygenated blood may be delivered through the second lumen, and fluids such as oxygen-depleted blood may be removed via the first lumen. Such operations will be described in greater detail below.

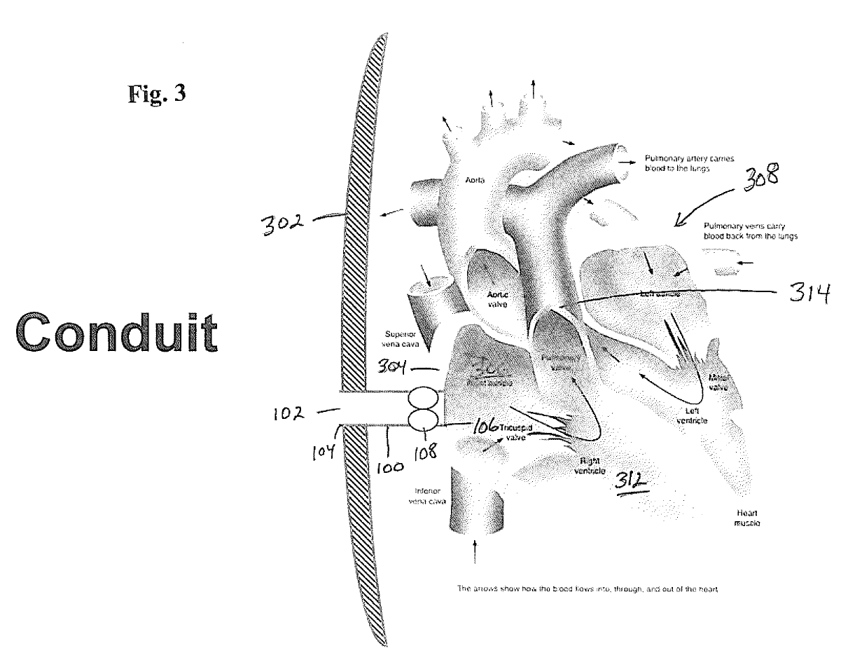

In use, the selectively occludable conduit 100 may be implanted in the chest wall of a patient (see FIG. 3) such that end 104 is anastomosed to the chest wall 302. End 106 is anastomosed to a wall 304 of a heart chamber 306, in the depicted embodiment being the right atrium, whereby fluid communication may be selectively established between the exterior of the chest wall 302 and the interior of the patient's heart 308. The allows in FIG. 3 depict the route of blood flow into and out of the heart 308. Bleeding is prevented by occluding at least end 106 of the conduit 100, such as by inflating occlusion balloons 108.

The above positioning of the selectively occludable conduit 100 provides an access conduit for various procedures relating to removal of fluid from and introduction of fluid into the heart 308. Typically, the fluid removed will be oxygen-depleted blood. The fluid introduced will typically be blood which has at least been oxygenated and had excess carbon dioxide removed, although other fluids are contemplated. Such procedures are well known in the medical arts.

Use of the conduit 100 and the described system 10 in one such procedure, a right ventricular assist procedure, will now be described. As is well known in the art, a right ventricular assist procedure is intended to aid the pumping action of a weakened heart right ventricle, either for shorter term support of a failing heart until a donor heart becomes available, or for longer term therapy in heart failure patients who are not candidates for heart transplant.

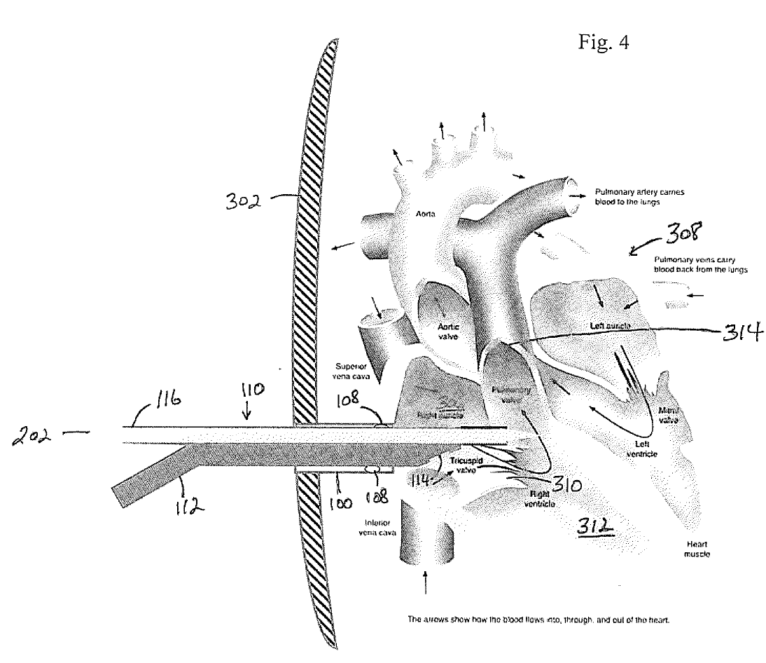

The dual lumen cannula 110, including an introducer 202, is inserted through lumen 102 of the selectively occludable conduit 100, with occlusion balloons 108 deflated (see FIG. 4). The cannula 110/introducer 202 are advanced until the distal end of the first lumen 112 is substantially positioned in an interior of the desired heart chamber 306 (in the embodiment shown, the right atrium 306, or substantially at a juncture of the right atrium 306, the right ventricle 312, the superior vena cava 311, and the inferior vena cava 313). The accurate positioning of the cannula 110 may be monitored by any suitable method, such as X-ray, ultrasound, and the like. In this position, blood may be removed from one or more of the right atrium 306, the right ventricle 312, the superior vena cava 311, and the inferior vena cava 313 through first lumen 112, via drainage aperture 114. The second lumen 116 is passed through the tricuspid valve 310 such that the distal end thereof is positioned near a juncture between the right ventricle 312 and the pulmonary artery 314. Occlusion balloons 108 may then be inflated to the degree necessary to prevent leakage of blood around an exterior of the cannula 110.

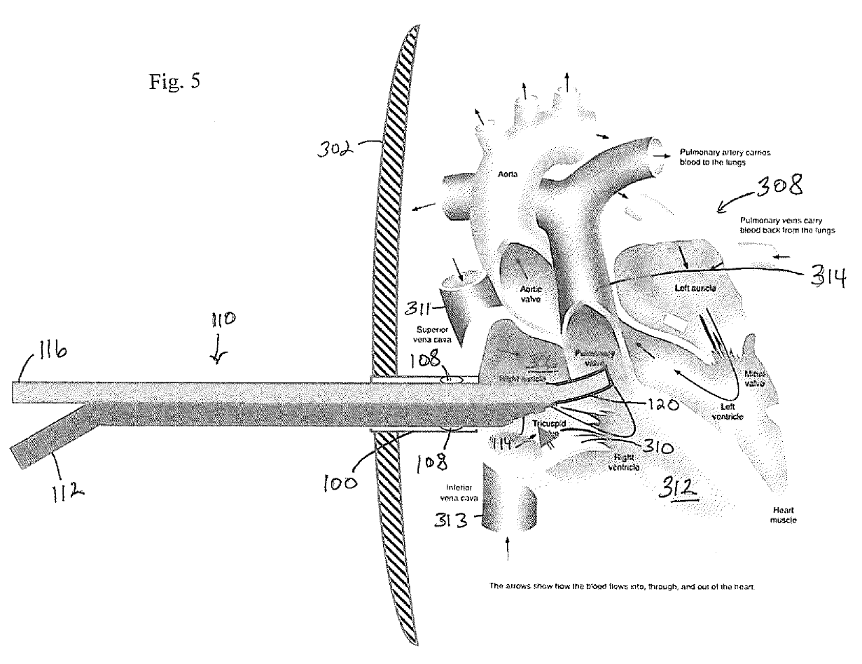

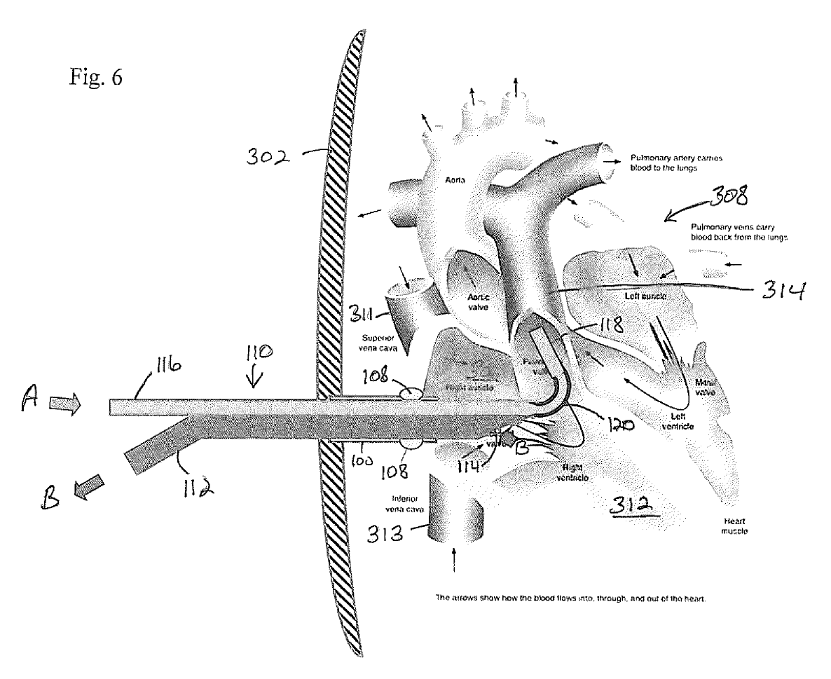

The introducer 202 is then removed (see FIG. 5) and the flexible portion 120 is caused to assume a predetermined angle or curvature sufficient to allow positioning the extendable infusion sleeve 118 at an opening of the pulmonary artery 314 for delivery of a fluid. Suitable mechanisms by which flexible portion 120 may be induced to assume that predetermined angle or curvature have been described above. Next, as shown in FIG. 6, extendable infusion sleeve 118 is advanced into an interior of the pulmonary artery 314, such as by passage of a fluid therethrough to dislodge the extendable infusion sleeve 118 from an interior of the selectively flexible portion 120. One or more extracoiporeal pumps (not shown) as are well known in the all for use in right ventricular assist devices (RVAD) operatively connected to the dual lumen cannula 110 allow introduction of oxygenated blood into the pulmonary artery (see arrows A) via second lumen 116 and removal of oxygen-depleted blood from a separate portion of the heart interior via first lumen 112. Suitable ports, connectors, and tubing as are well-known in the medical arts are contemplated for such operative connection to extracoiporeal ports. The skilled artisan will readily appreciate that this provides a means for by-passing the right ventricle in a patient having a failing right ventricle. Removal of blood from the right atrium 306 via first lumen 112 is contemplated, because of the reduced blood pressure typically found in the right atrium. Because of this reduced blood pressure, the risk of bleeding/blood leakage at the site at which conduit 100 is anastomosed to the heart chamber is reduced.

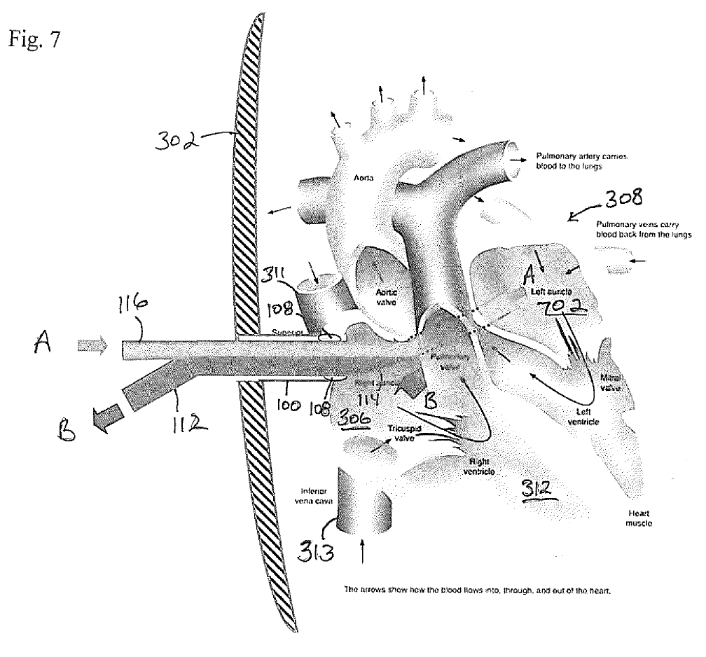

In another embodiment, the presently described selectively occludable conduit 100 may be used in an alternative procedure such as for artificial lung purposes. The selectively occludable conduit 100 is implanted substantially as described above. Next (see FIG. 7), the dual lumen cannula 110 having an atraumatic inducer 202 (not shown in this Figure) sleeved therein is passed through the conduit 100 substantially as described above, with the exception that a dual lumen cannula 10 is provided having a second lumen 116 which is of sufficient length that, after passage through the conduit 100, a distal end thereof is advanced into an interior of the left atrium 702 through an incision or puncture traversing the atrial septa (not shown for convenience).

After removal of the introducer 202 and operative connection of the dual lumen cannula 110 to one or more pumps (not shown) in the manner described above, it will be appreciated that oxygen-depleted blood may be withdrawn via first lumen 112 from the right atrium 306, and oxygenated blood may be delivered to the left atrium 702 via second lumen 116. Thus, an artificial lung function is established. In this embodiment of the dual lumen cannula 110, flexible portion 120 and extendable infusion sleeve 118 are unnecessary.

Of course, the skilled artisan will appreciate that the presently described dual lumen cannula 110 is suitable also for providing access to an interior of the heart via a percutaneous route, which as noted above may be desirable in certain situations. The depicted dual lumen cannula 110, with an atraumatic introducer 202 sleeved therein, may be advanced through an incision or puncture providing access to a jugular vein of the body, and therethrough into an interior of the heart, wherein the drainage aperture 114 of the first lumen 112 is disposed substantially at a juncture of the superior vena cava 311, die right atrium 306, the right ventricle 312, and the inferior vena cava 313. The precise positioning of the dual lumen cannula 110 may be monitored by any acceptable method, including without limitation X-ray, ultrasound, etc.

The introducer 202 is removed, and the selectively flexible portion 120 is caused to assume the predetermined angle or curvature as described above, whereby the extendable infusion sleeve 118 of second lumen 116 is disposed substantially at an opening of the pulmonary artery 314. Again, the desired positioning of the second lumen 116 may be visualized/confined by any suitable method. Passing a fluid through second lumen 116 causes tie extendable infusion sleeve 118 to extend into an interior of the pulmonary artery 314, for delivery of the fluid thereinto. This describes use of the dual lumen cannula 110 of the present invention in a right ventricular assist function, wherein the interior of the heart is accessed via a percutaneous route.

Similarly, an alternative embodiment of the dual lumen cannula 110 may be adapted for percutaneous access to an interior of the heart for an artificial lung function. The dual lumen cannula 110 having an atraumatic inducer 202 sleeved therein is passed through the jugular vein substantially as described above, with the exception that a dual lumen cannula 110 is provided having a second lumen 116 which is of sufficient length that, after the drainage opening 114 of the first lumen 112 is positioned substantially at a juncture of juncture of the superior vena cava 311, the right atrium 306, the right ventricle 312, and the inferior vena cava 313. The introducer 202 is removed, the selectively flexible portion 120 is caused to assume the predetermined angle or curvature as described, and the second lumen 116 is advanced into an interior of the left atrium 702 through an incision or puncture (not shown for convenience) traversing the atrial septum substantially as shown in FIG. 7.

After operative connection of the dual lumen cannula 110 to one or more pumps in the manner described above, it will be appreciated that oxygen-depleted blood may be withdrawn via first lumen 112 from at least one of the superior vena cava 311, the right atrium 306, the right ventricle 312, and the inferior vena cava 313, and oxygenated blood may be delivered to the left atrium 702 via second lumen 116 and extendable infusion sleeve 118. Thus, an artificial lung function is established.

There is accordingly provided a system 110 for providing access to an interior of a patient's heart which allows repeated access for procedures such as right ventricular assist and artificial lung procedures, and which may be incorporated into devices and systems for such procedures. Unlike conventional procedures, which require repeated implantation and removal of cannulae with associated risk of pain, sepsis, and the like, the present disclosure provides a substantially permanent or semi-permanent port (selectively occludable conduit 100) which allows such access without repeated implantation and removal of cannulae, such as in a blood vessel of the patient. Thus, patient comfort and mobility are improved, and risk of complicating factors such as sepsis and the like are substantially reduced. Alternatively, in situations where percutaneous access to the heart interior is more desirable, the present disclosure provides improved cannula designs allowing such percutaneous access.

One of ordinary skill in the art will recognize that additional embodiments of the invention are also possible without departing from the teachings herein. This detailed description, and particularly the specific details of the exemplary embodiments, is given primarily for clarity of understanding, and no unnecessary limitations are to be imported, for modifications will become obvious to those skilled in the art upon reading this disclosure and may be made without departing from the spirit or scope of the invention. Relatively apparent modifications, of course, include combining the various features of one or more figures or examples with the features of one or more of other figures or examples.

What is claimed is:

1. A system for providing access to an interior of a heart of a body for delivering and/or removing a fluid, comprising:
a selectively occludable conduit defining a lumen for providing access to an interior of at least one heart chamber from an exterior of a chest wall of the patient; and
a dual lumen cannula adapted for passing through an interior lumen of the conduit for delivering a fluid into and removing a fluid from the interior of the heart chamber.

2. The system of claim 1, wherein the selectively occludable conduit includes at least one balloon closure for substantially sealing an end of the conduit in fluid communication with the chamber of the heart.

3. The system of claim 1, wherein the dual lumen cannula includes a first, drainage lumen having a first length and including at least one distal drainage aperture for removing a fluid from the interior of the heart, and a second, delivery lumen for delivering a fluid into the interior of the heart, the second lumen having a length that is greater than the first lumen.

4. The system of claim 3, further including a closure for substantially occluding the distal drainage aperture, the closure being selectively translatable from a first, closed position to a second, open position.

5. The system of claim 3, wherein the second lumen includes a distal extendable infusion sleeve for delivering a fluid into an interior of die heart.

6. The system of claim 5, wherein the second lumen includes a selectively flexible portion which selectively assumes a predetermined angle or curvature for altering a path of travel of a fluid passed through the second lumen extendable infusion sleeve.

7. The system of claim 6, wherein the predetermined angle or curvature in the selectively flexible portion is created by one of a spring elbow, a flexible polymer, or a shape memory alloy.

8. The system of claim 7, wherein the predetermined angle or curvature is sufficient to deliver at least a distal end of the extendable infusion sleeve into an interior of a lumen of a pulmonary artery of the heart.

9. The system of claim 8, further including at least one pump for operatively connecting to the dual lumen cannula, for removing a fluid from the heart through the first lumen and for delivering a fluid to the heart through the second lumen.

10. A selectively occludable conduit for use in a right ventricular assist device or an artificial lung, comprising:
a body defining a lumen for providing fluid communication between an exterior of a patient chest wall of a patient and an interior of the patient heart; and
at least one selectively sealable closure for substantially preventing fluid communication between the exterior of the patient chest wall of a patient and the interior of the patient heart;
wherein a first end of the selectively occludable conduit is adapted to be anastomosed to a chest wall of the patient and a second, opposed end of the selectively occludable conduit is adapted to be anastomosed to the heart chamber for providing fluid communication to an interior of the heart chamber;
further wherein the at least one selectively sealable closure, when in a closed configuration, substantially prevents fluid communication between the chest wall exterior and the heart interior.

11. The selectively occludable conduit of claim 10, wherein the selectively sealable closure is an inflatable balloon closure.

12. A dual lumen cannula for delivering a fluid into and removing a fluid from an interior of a heart of a body, comprising:
a first drainage lumen having a first length and including at least one distal drainage aperture for removing a fluid from the interior of the heart chamber; and
a second delivery lumen for delivering a fluid into the interior of the heart chamber, the second lumen having a length that is greater than the first lumen.

13. The dual lumen cannula of claim 12, further including a closure for substantially occluding the distal drainage aperture, the closure being selectively translatable from a first, closed position to a second, open position.

14. The dual lumen cannula of claim 12, wherein the second lumen includes a distal extendable infusion sleeve for delivering a fluid into an interior of the heart.

15. The dual lumen cannula of claim 14, wherein the second lumen includes a selectively flexible portion which selectively assumes a predetermined angle or curvature for redirecting a path of travel of a fluid passed through the second lumen extendable infusion sleeve.

16. The dual lumen cannula of claim 15, wherein the predetermined angle or curvature in the selectively flexible position is created by at least one of a spring elbow, a flexible polymer, or a shape memory alloy.

17. The dual lumen cannula of claim 15, wherein the predetermined angle or curvature is sufficient to deliver at least a distal end of the extendable infusion sleeve into an interior of a lumen of a pulmonary artery of the heart.

18. A method for providing access to an interior of a patient heart for a right ventricular assist function or an artificial lung function, comprising:
providing a selectively occludable conduit defining a lumen, said selectively occludable conduit being adapted to be anastomosed at a first end to a chest wall of a patient and at a second, opposed end to a patient heart whereby selective fluid communication may be provided between an exterior of the patient chest wall and an interior of the heart;
passing a dual lumen cannula through the lumen of the selectively occludable conduit into the interior of the heart; and
operatively connecting the dual lumen cannula to at least one pump adapted for a right ventricular assist device or artificial lung, for removing a fluid from and delivering a fluid to the heart through the dual lumen cannula.

19. The method of claim 18, including providing a selectively occludable conduit having at least one inflatable balloon closure for preventing fluid communication between the exterior of die patient chest wall and the interior of the heart.

20. The method of claim 18, including providing a dual lumen cannula having a first, drainage lumen having a first length and including at least one drainage aperture for removing a fluid from an interior of the right atrium and a second, delivery lumen having second length for delivering a fluid into an interior of the heart, wherein the first length is less than the second length.

21. The method of claim 20, including providing a dual lumen cannula including a closure for substantially occluding the distal drainage aperture, the closure being selectively translatable from a first, closed position to a second, open position.

22. The method of claim 20, further including providing a dual lumen cannula wherein the second lumen includes a distal extendable infusion sleeve for delivering a fluid into an interior of the heart.

23. The method of claim 22, further including providing a dual lumen cannula including a second lumen having a selectively flexible portion for selectively assuming a predetermined angle or curvature sufficient to deliver at least a distal end of the extendable infusion sleeve into an interior of a lumen of a pulmonary artery of the heart.

24. The method of claim 23, including the steps of:
anastomosing the selectively occludable conduit at a first end to an exterior of a chest wall of a patient and at a second, opposed end to a patient heart whereby selective fluid communication may be provided between an exterior of the patient chest wall and an interior of a heart;
placing an atraumatic introducer in an interior of the dual lumen cannula second lumen and passing the dual lumen cannula and introducer through the conduit lumen into the interior of the heart right atrium;
withdrawing the atraumatic introducer and causing the selectively flexible portion to assume the predetermined angle or curvature;
extending the extendable infusion sleeve of the dual lumen cannula second lumen into an interior of a lumen of the pulmonary artery; and
operatively connecting at least one pump to the dual lumen cannula whereby oxygen-depleted blood is removed from an interior of the right atrium through the first lumen drainage aperture and oxygen-enriched blood is delivered to the interior of the pulmonary artery through the second lumen extendable infusion sleeve, thereby by-passing a heart right ventricle to perform a right ventricular assist function.

25. The method of claim 22, including the steps of:
anastomosing the selectively occludable conduit at a first end to an exterior of a chest wall of a patient and at a second, opposed end to a patient heart whereby selective fluid communication may be provided between an exterior of the patient chest wall and an interior of a heart right atrium;
providing an incision or puncture in a heart septa whereby fluid communication is established between the right atrium and a left atrium of the heart;
placing an atraumatic introducer in an interior of the dual lumen cannula second lumen and passing the dual lumen cannula and introducer through the conduit lumen into the interior of the heart right atrium;
positioning the dual lumen cannula whereby the drainage aperture of the first lumen of the dual lumen cannula is positioned in the interior of the light atrium and at least a distal end of the second lumen is passed through the incision or puncture into an interior of the left atrium;
withdrawing the atraumatic introducer; and
operatively connecting at least one pump to the dual lumen cannula whereby oxygen-depleted blood is removed from an interior of the right atrium through the first lumen and oxygen-enriched blood is delivered to the interior of the left atrium through the second lumen to perform an artificial lung function.

26. A method for providing access to an interior of a patient heart for a right ventricular assist function or an artificial lung function, comprising:
providing a dual lumen cannula having a first, drainage lumen having a first length and including at least one distal drainage aperture for removing a fluid from an interior of the right atrium and a second, delivery lumen having a second length for delivering a fluid into an interior of the heart, wherein the first length is less than the second length;
passing the dual lumen cannula through an incision or a puncture into a lumen of a jugular vein of the patient and therethrough into the interior of the heart; and
operatively connecting the dual lumen cannula to at least one pump adapted for a right ventricular assist device or artificial lung, for removing a fluid from and delivering a fluid to the heart through the dual lumen cannula.

27. The method of claim 26, including providing a dual lumen cannula including a closure for substantially occluding the distal drainage aperture, the closure being selectively translatable from a first, closed position to a second, open position.

28. The method of claim 26, further including providing a dual lumen cannula wherein the second lumen includes a distal extendable infusion sleeve for delivering a fluid into an interior of the heart.

29. The method of claim 28, further including providing a dual lumen cannula including a second lumen having a selectively flexible portion for assuming a predetermined angle or curvature sufficient to deliver at least a distal end of the extendable infusion sleeve into an interior of a lumen of a pulmonary artery of the heart.

30. The method of claim 29, including the steps of:
placing an atraumatic introducer in an interior of the dual lumen cannula second lumen and advancing the dual lumen cannula and introducer through the jugular vein lumen into the interior of the heart right atrium;
withdrawing the atraumatic introducer and causing the selectively flexible portion to assume the predetermined angle or curvature;
extending the extendable infusion sleeve of the dual lumen cannula second lumen into an interior of a lumen of the pulmonary artery; and
operatively connecting at least one pump to the dual lumen cannula whereby oxygen-depleted blood is removed from an interior of the right atrium through the first lumen drainage aperture and oxygen-enriched blood is delivered to the interior of the pulmonary artery through the second lumen extendable infusion sleeve, thereby by-passing a heart right ventricle to perform a light ventricular assist function.

31. The method of claim 29, including the steps of:
providing an incision or puncture in a heart septa whereby fluid communication is established between the right atrium and a left atrium of the heart;
placing an atraumatic introducer in an interior of the dual lumen cannula second lumen and passing the dual lumen cannula and introducer through the jugular vein lumen into the interior of the heart right atrium;
positioning the dual lumen cannula whereby the drainage aperture of the first lumen of the dual lumen cannula is positioned in the interior of the right atrium;
withdrawing the atraumatic introducer and causing the selectively flexible portion to assume the predetermined angle or curvature whereby at least a distal end of the second lumen is passed through the incision or puncture into an interior of the left atrium; and
operatively connecting at least one pump to the dual lumen cannula whereby oxygen-depleted blood is removed from an interior of the right atrium through the first lumen and oxygen-enriched blood is delivered to the interior of the left atrium through the second lumen to perform an artificial lung function.
